(19) United States
(12) Patent Application Publication
(10) Pub. No.: US 2009/0163442 A1
(43) Pub. Date: Jun. 25, 2009

(54) TREATMENT OF CANCER WITH 2-DEOXYGALACTOSE

(76) Inventors: Bernard R. Landau, Cambridge, MA (US); Steven Landau, Wellesley, MA (US)

Correspondence Address: Edwards Angell Palmer & Dodge LLP, P.O. Box 55874, Boston, MA, 02205, US

(21) Appl. No.: 12/196,195

(22) Filed: Aug. 21, 2008

(30) Foreign Application Priority Data
Feb. 21, 2007 (US) PCT/US2007/004465

(51) Int. Cl. A61K 31/702; A61K 31/7004; A61P 35/00; A61K 31/7016; C07H 3/04; C07H 3/06

(52) U.S. Cl. 514/61; 514/23; 514/54; 536/123.1; 536/123.13

(57) ABSTRACT

2-Deoxygalactose and its analogs can be used to treat cancer and to improve patient outcome when administered in therapeutically effective doses alone or in combination with other anti-cancer drugs or with surgical resection or radiation therapy.

### CROSS-REFERENCE TO RELATED APPLICATIONS

The present application claims the benefit of priority under 35 U.S.C. § 119(e) to U.S. Provisional Patent Application No. 60/775,630, entitled Treatment of Cancer with 2-Deoxygalactose, filed Feb. 21, 2006, the disclosure of which is incorporated herein by reference in its entirety.

### BACKGROUND OF THE INVENTION

The term “cancer” generally refers to one of a group of more than 100 diseases caused by the uncontrolled growth and spread of abnormal cells that can take the form of solid tumors, lymphomas, and non-solid cancers such as leukemia. Normal cells reproduce until maturation is attained and then only as necessary for replacement. Benign tumors, or benign hyperplasia, involve an overgrowth of cells without spread to other organs. Cancer cells, conversely, grow and divide endlessly, crowding out nearby cells and eventually spreading to other parts of the body. Cancer cells that develop at one site can grow rapidly into a malignant tumor, invading and destroying nearby tissues. Malignant cancer tumor cells eventually metastasize, or spread to other parts of the body via the bloodstream or lymphatic system, where the cells begin multiplying and developing into new tumors. This sort of tumor progression makes cancer dangerously fatal. Although there have been great improvements in diagnosis, general patient care, surgical techniques, and local and systemic adjuvant therapies, most deaths from cancer are still due to metastases and resistance to conventional therapies.

The vast majority of drug-mediated conventional cancer therapies rely on the use of drugs that act as selective poisons for dividing cells. These drugs are effective, because cancer cells generally divide more frequently than normal cells. However, for a variety of reasons, such drugs almost inevitably do not kill all cells in a tumor. One reason is that not all cancer cells divide more frequently than all normal cells. Another is that specific proteins can confer drug resistance to a cancer cell, creating multiple drug resistance (“M-DR”) phenotype. Another is the very nature of the tumor, particularly its vascular architecture. As a tumor grows, it requires a blood supply and growth of new vasculature. The new vasculature that supports the tumor growth is often disordered, leaving significant portions of the tumor under-vascularized and even the vascularized portions subject to intermittent vascular blockage. The vasculature delivers not only oxygen but also most anti-cancer drugs to cells. Thus, the hypoxic regions of tumors are typically under-supplied with anti-cancer drugs. Oxygen is critical for supplying energy to a cell in the form of ATP produced by mitochondrial action, and cell growth and division are energy intensive processes. In addition, oxygen is required for the cytotoxic action of some anti-cancer drugs and radiation therapies. A cell's only other source of ATP in the amounts needed to support the cell is from anaerobic glycolysis. Given the demand for ATP caused by cell division and the hypoxic nature of tumors, it is therefore not surprising that many cancers exhibit, relative to normal cells, increased glucose transport and glycolysis. This attribute of cancer cells was described, for example, in Dickens, 1943, Cancer Research 3:73, which reported “the typical intact cancer cell exhibits an unusual ability to utilize glucose by the process of anaerobic glycolysis through lactate”.

Today, this unusual ability of cancer cells to utilize glucose is exploited to image tumors in the diagnostic technique of PET scanning, which utilizes a radioactively labeled glucose analog 18F-2-deoxy-D-glucose (“FDG”) that preferentially accumulates in cancer cells relative to most normal cells. See Som et al., 1980, “A fluorinated glucose analog, 2fluoro-2-deoxy-D-glucose (F-18): nontoxic tracer for rapid tumor detection”, J. Nucl. Med. 21: 670-675. Scientists have questioned whether the increased glycolysis in cancer cells relative to normal cells would also allow the use of metabolic poisons of anaerobic glycolysis to target cancer cells preferentially. 2-Deoxy-D-glucose (“2-DG”; see Bergmann, 1922, Deutsch. Chem. Ges. 56:158-60; Cramer, 1952, Franklin Inst. 253:277-80; Japan patent publication No. 54-041384) is a metabolic poison (see McDonald, 1952, Cancer Research 351-353) that inhibits glycolysis in cancer cells (see Woodward, 1954, Cancer Res. 14:599605).

2-DG is believed to inhibit glycolysis by accumulating in the cell, into which it is transported by one or more glucose transporters. Once in the cell, hexokinase converts 2-DG to 2-DG-6-phosphate (“2DG6P”), which cannot be converted to fructose-6-phosphate, a substrate required for glycolysis, and which cannot leave the cell unless the phosphate group is removed by glucose-6-phosphatase (abundant in liver cells) or non-specific, intracellular phosphatases. When intracellular concentrations of 2DG6P reach a certain threshold amount, glycolysis is effectively shut down, and the cell dies from a lack of energy in the form of ATP. 2-DG may have other cytotoxic effects as well. It can act as a trap for phosphate and so reduce intracellular ATP, first in its conversion to 2DG6P and then its conversion to uridyl diphosphate-2-DG (“UDP2DG”). The formation of the latter compound UDP2DG acts as a trap for uridyl, thus inhibiting nucleic acid synthesis. In addition, 2DG6P may inhibit the pentose cycle that generates ribose and deoxyribose and so could inhibit DNA synthesis even beyond the inhibition caused by the phosphate, ATP, and uridyl trapping attendant upon 2DC uptake by a cell. There is some evidence that 2-DG can be converted to 2-DG-1phosphate (“2DG1P’; see Colwell et al., 1996, Int. J. Biochem. Cell. Biol. 28(1): 115-121) and then be incorporated into glycogen. In addition, there is evidence that 2-DG is incorporated into glycolipids and glycoproteins (via UDP2DG) and so interfere with their normal processing and function (see Steiner et al., 1974, Biochem. Biophys. Res. Comm. 61(2): 795).

2-DG has been shown to retard tumor growth in animal models (see Sokoloff, 1955, A.M.A. Arch. Of Path. 729-732, and Ball, 1957, Cancer Res. 17:235-39) and was administered to humans as early as the 1950s (see Landau, 1958, J. Natl. Conc. Inst. 21:485-494). Cell-based, animal, and human studies of 2-DG conducted since the 1950s have, with some exceptions, continued to indicate that 2-DG has an impact on cancer cells, alone or in combination with radiation or other chemotherapy. Thus, Laszlo et al., February 1960, J. Natl. Cane. Inst. 24(2):267-281, reported cancer studies in mice in which “some evidence of interference with the normal course of the diseases, either by impairment of local tumor growth or by prolongation of host survival, or both” was seen. See also Haberkorn, November 1992, J. Nucl. Med. 33(11):1981-87, and Malaisse, March 1998, Cancer Lett. 125:45-49. Likewise, Purohit, March 1982, Int. J. Radiat. Oncol. Biol. Phys. 8:495-99, reported 2-DG inhibited cancer cell growth, with more pronounced inhibition under hypoxic conditions and in combination with X-irradiation. See also Dwarakanath, March 1999, Int. J. Radiat. Oncol. Biol. Phys. 43(5):1125-33; Dwarakanath, Jul. 2001, Int. J. Radiant. Oncol Biol. Phys. 50(4):1051-61; and Yeung, 11 Dec. 2001, PCT WO 02/58741, which reports that ‘⁸F-2-DG could be administered to treat cancer, at doses significantly higher than those used for diagnostic imaging. Combination studies of 2-DG with other cytotoxins and anti-cancer drugs include those described in Lampidis, 2 Mar. 2001, PCT WO 01/82926, which states that 2DG, oxamate, and various analogs are selectively toxic toward anaerobic cells and can be used to increase the efficacy of standard cancer chemotherapeutic and radiation regimens. The reference Pitha, 21 Mar. 200.2, U.S. patent publication No. 20020035071, discloses a set of compounds that purportedly mimic the effect of 2-DG without the toxicities attributed to that compound (including hunger, sweating, ataxia, and convulsions).

2-Deoxy-D-galactose (“2-DGal”) can be considered an analog of 2-DG, in that it has a closely related structure, differing only in the position of the hydroxyl group at C-4.2DGal can in fact be converted in the body to 2-DG by a process that involves conversion of 2-DGal first to 2-DGal-1-phosphate, then to uridyl diphosphate 2-DGal (“UDP2DGal”), then to UDP2DG, then to 2-DG-1-phosphate, then to 2-DG-6-phosphate, and finally to 2-DG. This bioconversion closely follows the pathways for the natural sugars galactose and glucose. Galactose is metabolized in the glycolytic pathway by conversion first to galactose-1phosphate, then to uridine diphosphate galactose (“UDPGaI”), then to UDP-glucose (“UDPGIu”; the precursor to glycogen), then to glucose-1-phosphate, and then to glucose-6phosphate, the initial substrate in the glycolytic pathway. However, galactose and glucose have very different roles in the body and are metabolized quite differently in some respects. First, galactose is metabolized first by galactokinase to form galactose-1-phosphate, whereas glucose is metabolized first by hexokinase to glucose-6-phosphate. Second UDPGaI is a direct substrate used in the glycosylation of proteins; UDPGlu is not. Third, the liver is the major organ of galactose metabolism, and galactose is cleared rapidly from the blood so that, if galactose is ingested, the concentration entering the liver via the portal vein is much higher than that leaving via the hepatic veil. Fourth, the accumulation of galactose-1-phosphate results in the clinical symptoms of the disease condition known as galactosemia, which is fatal unless galactose is avoided and can have severe long term complications even so.

In other respects, 2-DG and 2-DGaI are similar. Both compounds are actively transported across membranes in insulin responsive tissues (see Landau et al, June 1958, Aim. J. Physiol. 193(3). 461-465). Neither compound is actively transported by the intestine, although passive diffusion clearly enables absorption of large amounts of the compounds (see Wilson and Landau, 1960, Am. J. Physiol. 198: 99-102). Both 2-DG and 2-DGal have been shown to inhibit glycolysis in tumor cell lines and types (see Laszlo et al., September 1958, Natl. Cancer Inst. 21(3): 475-483). In addition, both 2-DG and 2-DGal were reported to cause “inhibition of tumor growth . . . and a modest prolongation of survival time . . . ” in animal studies, although the results reported indicated that 2-DGal was less efficacious in the model systems employed (see Laszlo et al., February 1960, J. Natl. Canc. Inst. 24(2):267-281, supra). Interestingly as well, the 2-fluoro-derivative of 2-DGal has been reported to have properties that might make it a useful cancer imaging and/or therapeutic agent (see Ishiwata et al., 1989, Nucl. Med. Biol. 16(3): 247-254; Grun et al., 1990, Adv. Enzyme Regul. 30: 231-242; Grun et al., 31 May 1990, Eur. J. Biochem. 190(1). 11-19; Fukuda et al., 1986, Eur. J. Nucl. Med. 11(11): 444-448; and Paul et al., 1989, Int. J. Rad. Appl. Instrum. B. 16(5): 449-453). Also, 2-DGaI has been shown to trap phosphorous and uridylate and to block glycolysis, all leading to a depletion of ATP, in cancer cells (see Smith and Keppler, 1977, Eur. J. Biochem. 73: 8392, and Keppler et al., 1985, Adv. Enz. Reg. 23: 61-79).

However, after more than five decades of study, the benefits of 2-DG or analogs thereof such as 2-DGal in cancer therapy are still uncertain, and neither 2-DG, 2-DGaI, nor any analog compound has been approved for the treatment of cancer in the United States or Europe. Given the many reported studies that suggest 2-DG and its analogs may have a role to play in cancer therapy, perhaps in combination with other treatment regimens, there remains a need for methods of treating cancer with 2-DG or its analogs. The present invention meets that and other needs.

### SUMMARY OF THE INVENTION

In a first aspect, the present invention provides a method of treating cancer, which method comprises administering to a mammal a therapeutically effective dose of 2-DGaI or a 2-DGaI analog. In one embodiment, the therapeutically effective dose is a dose in the range of about 1 mg/kg (patient weight) to about 5g/kg of 2-DGal or a 2-DGaI analog. In another embodiment, the therapeutically effective dose is a dose in the range of about 10 mg/kg to about 1g/kg. In another embodiment, the therapeutically effective dose is about 50 mg/kg to about 500 mg/kg. In one embodiment, 2-DGal or a 2-DGal analog is administered in a single oral dose of from about 5 to about 25 grams. In one embodiment, the 2-DGal or 2-DGal analog is administered orally once (qday), twice (bid), three times (tid), or four times (qid) a day or once every other day (qod) or once a week (qweek), and treatment is continued for a period ranging from three days to two weeks or longer. In one embodiment, the treatment is continued for one to three months. In another embodiment, the treatment is continued for a year.

In a second aspect, the present invention provides a pharmaceutically acceptable formulation of 2-DGal and 2-DGal analog useful in the methods of the present invention. In one embodiment, the formulation is crystalline in nature, and the 2-DGaI or 2-DGal analog is packaged in a sachet that is decanted into and dissolved in a liquid for oral administration to the patient. In other formulations, the 2-DGal or 2-DGaI analog is not crystalline but can be amorphous in nature. In another embodiment, the 2-DGaI or 2-DGaI analog is formulated as a tablet or pill containing 2-DGal or a 2-DaI analog in an amount in the range of about 250 mg to about 2g.

In a third aspect, the present invention provides a method of treating or preventing the reoccurrence of liver or brain cancer, which method comprises administering to a mammal a therapeutically effective dose of 2-DGaI or a 2-DGal analog.

In a fourth aspect, the present invention provides a method of treating or preventing cancer, which method comprises administering to a human or other mammal a therapeutically effective dose of 2-DGal or a 2-DGaI analog in combination with another anti-cancer agent and/or radiation therapy. In one embodiment, the cancer is a liver cancer, a brain cancer, or a multi-drug resistant cancer or a cancer that is otherwise refractory to treatment. In one embodiment, the cancer is a brain cancer, and the 2-DGaI is administered concurrently with radiation therapy. In another embodiment, the 2-DGaI or 2-DGaI analog is co-administered with one or more of the following agents: 2-deoxy-D-glucose, 3-bromopyruvate, and a cytotoxic agent.

These and other aspects and embodiments of the invention are described in more detail in the detailed description, examples, and claims that follow.

### DETAILED DESCRIPTION OF THE INVENTION

The present invention provides methods of treating cancer by administering a therapeutically effective dose of 2-DGaI or a 2-DGal analog, alone or in combination with other anti-cancer therapies, including surgical resection, radiation therapy, and drug therapy. To aid in the appreciation of the invention, this description is divided into the following topics: (i) therapeutically effective administration of 2-DGal or a 2-DGaI analog; (ii) co-administration with other anti-cancer agents; (iii) treating particular cancers; and (iv) formulation and packaging of a 2-DGaI and 2-DGal analogs.

### Therapeutically Effective Administration of 2-DGal and 2-DGaI Analogs

The present invention provides methods and compositions for the treatment of cancer with 2-DGaI and 2-DGaI analogs. In accordance with the methods of the present invention, a 2-DGaI analog is any D-galactose analog other than 2-DGal that does not have a hydroxyl group at the 2 position of the galactose ring and that is capable of being phosphorylated in vivo in mammalian cells when present in the cell in monomer form. Lgalactose and its L-analogs are not 2-DGal analogs for purposes of the present invention. A galactose analog includes D-lyxo-hexos-2-ulose, D-arabino-hexos-2-ulose, and 5-thio-galactose. An analog of galactose or 2-DGaI can have a fluorine in place of a hydrogen at any position on the galactose ring; thus, 2-fluoro-2-deoxy-D-galactose (2-FDGa1) and 2-difluoro2-deoxy-D-galactose are 2-DGal analogs. An analog of galactose or 2-DGal can have an amino group in place of a hydroxyl group at any position on the glucose ring other than the 6 position; thus, 2-amino-2-deoxy-D-galactose (2-galactosamine) is a 2-DGaI analog. Other illustrative 2-DGaI analogs include di, tri, and other oligosaccharides that contain 2-DGaI and/or a 2-DGaI analog.

In accordance with the methods of the present invention, 2-DGaI or a 2-DgaI analog must be administered in a dose in the range of about 1 mg to about 1 g of 2-DGaI or a 2-DGaI analog per kg of body weight of the patient to be treated, and more than one dose must be administered. While the patient will most often be a human patient, those of skill in the art will appreciate that the methods and compositions of the invention can be used to treat cancer in any mammal. In one embodiment, the 2-DGaI or 2-DGaI analog is administered in a dose in the range of about 10 mg to about 750 mg of 2-DGaI or 2-DGal analog per kg of body weight of the patient to be treated. In another embodiment, the 2-DGaI or 2-DGaI analog is administered in a dose in the range of about 100 mg to about 500 mg of 2-DGaI or 2-DGal analog per kg of body weight of the patient to be treated. In certain other embodiments, the 2-DGaI or 2-DGaI analog is administered in a dose of about 50 to 250 mg of 2-DGaI or 2-DGal analog per kg of body weight of the patient to be treated. In one embodiment, from about 10 to about 25g of 2-DGaI or a 2-DGaI analog are administered orally per day (in one or more doses) for the treatment of cancer.

The therapeutically effective dose of 2-DGal or a 2-DGal analog is administered daily, or once every other day, or once a week to the patient, and multiple administrations of the drug are employed. Depending on the dose selected by the practitioner and the convenience of the patient, the entire dose may be administered once daily; or the dose may be administered in multiple smaller doses through the course of a day. For example, the dose may be divided into two smaller doses and administered twice daily, or divided into three smaller doses and administered thrice daily. Alternatively, the dose may be combined and given every other day, or even less frequently, but in any event, the dose is repeatedly administered over a period of time.

For optimum treatment benefit, the administration of the therapeutically effective dose is continued for multiple days, typically for at least five consecutive days, and often for at least a week and often for several weeks or more. Thus, a patient may be administered 2-DGal or a 2-DGaI analog in accordance with the present methods for a week, a month, two months, three months, six months, or a year or longer. For preventive applications, treatment may continue indefinitely throughout the life of the patient. As is well understood in the art for other cancer therapeutic drugs, treatment with 2-DGaI or a 2-DGaI analog may be suspended temporarily if toxicity is observed or for the convenience of the patient without departing from the scope of the invention. In re-treatment regimens, the dose is adjusted to reflect patient tolerance of the prior treatment.

In one preferred embodiment of the invention, the 2-DGal or a 2-DGaI analog is administered orally, and multiple doses are administered over an extended period of time. Using this therapeutically effective dosing and administration regimen, practitioners of skill in the alt can significantly improve treatment outcomes with all currently used cancer therapies, including surgical resection, radiation therapy, and drug therapies. In one important aspect, the present invention provides new methods for treating cancer using existing anti-cancer drugs as well as other compounds, as discussed in the following section.

Co-Administration with Other Anti-Cancer Agents

In accordance with the methods of the invention, 2-DGal or a 2-DGal analog can be co-administered in combination with other anti-cancer and antineoplastic agents.

In some embodiments, 2-DGaI or a 2-DGaI analog is administered prior to the initiation of any other cancer therapy, and treatment is continued throughout the course of the other therapy. In other embodiments, 2-DGal or a 2-DGaI analog is administered after the initiation or completion of the other cancer therapy. In other embodiments, 2-DGaI or a 2DGaI analog is first administered at the initiation of the other cancer therapy.

In one important embodiment, 2-DGaI or a 2-DGaI analog is administered with another anti-cancer agent that is more effective when ATP levels in the cancer cell are low. 2DGaI and 2-DGaI analogs act in part by reducing the ATP available to the cancer cell. Thus, in one aspect of the invention, 2-DGaI or a 2-DGal analog is administered once in an amount effective for reducing ATP levels in the tumor and administered again only after ATP levels begin to rise again; thereafter, 2-DGaI or a 2-DGaI analog is administered to maintain ATP at a low level in the tumor. As but one example, the DNA damage induced by radiation therapy and by certain drug therapies requires ATP to repair. Consequently, administration of 2-DGal or a 2-DGaI analog in accordance with the methods of the present invention can improve patient outcomes when conducted concurrently with such therapies. In one embodiment, the present invention provides a method of treating brain cancer, which method comprises administering a therapeutically effective dose of 2-DGal or a 2-DGaI analog as described herein in combination with a radiation therapy regimen that has been used for the treatment of brain cancer.

In addition to the combination of 2-DGal or a 2-DGaI analog with agents that are more effective when ATP levels are reduced, the present invention provides a variety of synergistic combinations of 2-DGal or a 2-DGaI analog and other anticancer drugs. Those of skill in the art can readily determine in accordance with the invention what anti-cancer drugs can be used “synergistically” with 2-DGal or a 2-DGal analog. For example, the reference Vendetti, “Relevance of Transplantable Animal-Tumor Systems to the Selection of New Agents for Clinical Trial,” Pharmacological Basis of Cancer Chemotherapy, Williams and Wilkins, Baltimore, 1975, and Simpson Herren et al., 1985, “Evaluation of In Vivo Tumor Models for Predicting Clinical Activity for Anticancer Drugs,” Proc. Am. Assoc. Cancer Res. 26: 330, each of which is incorporated herein by reference, describe methods to aid in the determination of whether two drugs act synergistically. While synergy is not required for therapeutic benefit in accordance with the methods of the invention, synergy is preferred, and two drugs can be said to possess therapeutic synergy if a combination dose regimen of the two drugs produces a significantly better tumor cell kill than either of the single agents at optimal or maximum tolerated doses. The “degree of synergy” can be defined as net logs of tumor cell kill by the optimum combination regimen minus net logs of tumor cell kill by the optimal dose of the most active single agent. Differences in cell kill of greater than ten-fold (one log) are considered conclusively indicative of therapeutic synergy.

Even where 2-DGal or a 2-DGal analog acts synergistically or merely additively with another anti-cancer agent, 2-DGaI or a 2-DGal analog will, at least in some embodiments, be administered prior to the initiation of therapy with the other drug or drugs and will, in any event, typically continue throughout the course of treatment with the other drug or drugs. In a related aspect, the present invention also provides new methods of using known anti-cancer therapies, particularly drug-based anti-cancer therapies, in which 2-DgaI or a 2-DGaI analog is administered concurrently with a therapy in which the active agent is delivered at a lower dose, and optionally for longer periods, than is currently practiced. Such “low dose” therapies can involve, for example, administering an anti-cancer drug, including but not limited to paclitaxel, doxorubicin, cisplatin, or carboplatin, at a lower than approved dose and for a longer period of time together with 2-DGal or a 2-DGaI analog administered in accordance with the methods of the present invention. These methods can be used to improve patient outcomes over currently practiced therapies by more effectively killing cancer cells or stopping cancer cell growth as well as diminishing unwanted side effects of the other therapy.

When employed in combination with one of these agents, the dosages of the additional agent are either the standard dosages employed for those agents or are adjusted downward from levels employed when that agent is used alone. Thus, the administration of a 2-DGal or a 2-DGaI analog in accordance with the methods of the invention can allow the physician to treat cancer with existing drugs, but at a lower concentration or dose than is currently used, thus ameliorating the toxic side effects of such drugs. The determination of the exact dosages for a given patient varies, dependent upon a number of factors including the drug combination employed, the particular disease being treated, and the condition and prior history of the patient, but is within the skill of the ordinarily skilled artisan in view of the teachings herein.

Specific dose regimens for known and approved anti-neoplastic agents are given, for example, in the product descriptions found in the current edition of the Physician's Desk Reference, Medical Economics Company, Inc., Oradell, N.J. Illustrative dosage regimens for certain anti-cancer drugs are also provided below. Those of skill in the art will recognize that many of the known anti-cancer drugs discussed herein are routinely used in combination with other drugs. In accordance with the methods of the present invention, 2-DGal or a 2-DGaI analog can be co-administered in such multiple drug treatment regimens.

Cancer drugs can be classified generally as alkylators, anthracyclines, antibiotics, aromatase inhibitors, biphosphonates, cyclo-oxygenase inhibitors, estrogen receptor modulators, folate antagonists, inorganic arsenates, microtubule inhibitors, metabolic inhibitors, modifiers, nitrosoureas, nucleoside analogs, osteoclast inhibitors, platinum containing compounds, retinoids, topoisomerase 1 inhibitors, topoisomerase 2 inhibitors, and tyrosine kinase inhibitors. In accordance with the methods of the present invention, 2-DGaI or a 2-DGaI analog can be co-administered with any anti-cancer drug from any of these classes or can be administered prior to or after treatment with any such drug or combination of such drugs. In one embodiment, however, the anti-cancer drug co-administered with 2DGal or a 2-DGaI analog is not a topoisomerase inhibitor.

Alkylators useful in the practice of the present invention include but are not limited to busulfan (Myleran, Busulfex), chlorambucil (Leukeran), cyclophosphamide (Cytoxan, Neosar), melphalan, L-PAM (Alkeran), dacarbazine (DTIC-Dome), and temozolamide (Temodar). In accordance with the methods of the present invention 2-DGal or a 2-DGaI analog is co-administered with an alkylator to treat cancer. In one embodiment, the cancer is chronic myelogenous leukemia, multiple myeloma, or anaplastic astrocytoma. As one example, the compound 2-bis[(2-Chloroethyl)amino]tetrahydro-2H-1,3,2-oxazaphosphorine, 2-oxide, also commonly known as cyclophosphamide, is an alkylator used in the treatment of Stages III and IV malignant lymphomas, multiple myeloma, leukemia, mycosis fungoides, neuroblastoma, ovarian adenocarcinoma, retinoblastoma, and carcinoma of the breast. Cyclophosphamide is administered for induction therapy in doses of 1500-1800 mg/m² that are administered intravenously in divided doses over a period of three to five days; for maintenance therapy, 350-550 mg/m² are administered every 7-10 days, or 110-185 mg/m² are administered intravenously twice weekly. In accordance with the methods of the invention, 2-DGal or a 2-DGaI analog is co-administered with cyclosphosphamide administered at such doses.

Anthracyclines useful in the practice of the present invention include but are not limited to doxorubicin (Adriamycin, Doxil, Rubex), mitoxantrone (Novantrone), idarubicin (Idamycin), vdlrubicin (Valstar), and epirubicin (Ellence). In accordance with the methods of the present invention 2-DGaI or a 2-DGal analog is co-administered with an anthracycline to treat cancer. In one embodiment, the cancer is acute nonlymphocytic leukemia, Kaposi's sarcoma, prostate cancer, bladder cancer, metastatic carcinoma of the ovary, and breast cancer. As one example the compound (8S,10S)-10-[(3-Amino-2,3,6-trideoxy-.alpha.-L-lyxohexopyranosyl)oxy]-8-glycoloyl-7,8,9,10-tetrahydro-6,8,11-trihydroxy-1-methoxy-5,12naphthacencdione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumor, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma. Doxorubicin is typically administered in a dose in the range of 30-75 mg/m² as a single intravenous injection administered at 21-day intervals; weekly intravenous injection at doses of 20 mg/m²; or 30 mg/m² doses on each of three successive days repeated every four weeks. In accordance with the methods of the invention, 2-DGal or a 2-DGal analog is co-administered starting prior to and continuing after the administration of doxorubicin at such doses.

Antibiotics useful in the practice of the present invention include but are not limited to dactinomycin, actinomycin D (Cosmegen), bleomycin (Blenoxane), and daunorubicin, daunomycin (Ceruibidine, DanuoXome). In accordance with the methods of the present invention 2-DGaI or a 2-DGal analog is co-administered with an antibiotic to treat cancer. In one embodiment, the cancer is acute lymphocytic leukemia, other leukemias, and Kaposi's sarcoma.

Aromatase inhibitors useful in the practice of the present invention include but are not limited to anastrozole (Arimidex) and letroazole (Femara), In accordance with the methods of the present invention 2-DGaI or a 2-DGaI analog is co-administered with an aromatase inhibitor to treat cancer. In one embodiment, the cancer is breast cancer.

Biphosphonate inhibitors useful in the practice of the present invention include but are not limited to zoledronate (Zometa). In accordance with the methods of the present invention 2-DGal or a 2-DGal analog is co-administered with a biphosphonate inhibitor to treat cancer. In one embodiment, the cancer is multiple myeloma, bone metastases from solid tumors, or prostate cancer.

Cyclo-oxygenase inhibitors useful in the practice of the present invention include but are not limited to celecoxib (Celebrex). In accordance with the methods of the present invention 2-DGal or a 2-DGaI analog is co-administered with a cyclo-oxygenase inhibitor to treat cancer. In one embodiment, the cancer is colon cancer or a pre-cancerous condition known as familial adenomatous polyposis.

Estrogen receptor modulators useful in the practice of the present invention include but are not limited to tamoxifen (Nolvadex) and fulvestrant (Faslodex). In accordance with the methods of the present invention 2-DGal or a 2-DGal analog is co-administered with an estrogen receptor modulator to treat cancer. In one embodiment, the cancer is breast cancer or the treatment is administered to prevent the occurrence or reoccurrence of breast cancer.

Folate antagonists useful in the practice of the present invention include but are not limited to methotrexate and tremetrexate. In accordance with the methods of the present invention 2-DGaI or a 2-DGaI analog is co-administered with a folate antagonist to treat cancer. In one embodiment, the cancer is osteosarcoma. Antifolate drugs have been used in cancer chemotherapy for over thirty years. As one example, the compound N-[4-[[(2,4diamino-6-pteridinyl)methyl methylamino]benzoyl]-L-glutamic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides. 5-Methyl-6-[[(3,4,5trimethoxyphenyl)-amino]methyl]-2,4-quinazolinediamine is another antifolate drug and is commonly known as trimetrexate. Methotrexate is administered as follows. For choriocarcinoma, intramuscular injections of doses of 15 to 30 mg daily for a five-day course, such courses repeated as needed with rest period of one or more weeks interposed between courses of therapy. For leukemias, twice weekly intramuscular injections in doses of 30 mg/m². For mycosis fungoides, weekly intramuscular injections of doses of 50 mg or, alternatively, of 25 mg twice weekly. In accordance with the methods of the invention, 2DGaI or a 2-DGaI analog is co-administered with methotrexate administered at such doses.

Inorganic arsenates useful in the practice of the present invention include but are not limited to arsenic trioxide (Trisenox). In accordance with the methods of the present invention 2-DGal or a 2-DGaI analog is co-administered with an inorganic arsenate to treat cancer. In one embodiment, the cancer is refractory APL.

Metabolic inhibitors useful in the practice of the present invention include any agent that interferes with glycolysis, such as, for example, 2-deoxy-D-glucose; 2-fluoro-2-deoxy-Dglucose; 2-deoxyfructose; lonidamine; and 3-broinopyruvate. In accordance with the methods of the present invention 2-DGal or a 2-DGal analog is co-administered with a metabolic inhibitor, alone or in combination with another cytotoxic or other anti-cancer agent, to treat cancer. The present invention also provides novel compounds of the invention and pharmaceutical compositions comprising such compounds useful in these methods.

Thus, in one embodiment, the present invention provides disaccharides, trisaccharides, and longer oligosaccharides comprising at least a 2-DGaI or a 2-DGaI analog and another pentose or hexose sugar, including but not limited to a pentose or hexose sugar that is itself an inhibitor of glycolysis or of DNA synthesis or some other aspect of nucleotide metabolism. In the trisaccharides and longer oligosaccharides, the 2-DGaI or 2-DGaI analog content can be varied, such as, for example 33% or 66%, as may be convenient for optimal delivery. The linkages of the monosaccharides to one another in such disaccharides, trisaccharides, and longer oligosaccharides can include at least one 1,4 linkage, such as in 2DGal-beta(1,4)-2-DG, that is cleavable by lactase. Other novel compounds of the invention are conjugates of 2-DGaI or a 2-DGal analog and 3-bromopyruvate joined by an ester linkage. Other novel compounds of the invention that are useful metabolic inhibitors alone or in combination with 2-DGal or a 2-DGaI analog include a disaccharide, trisaccharide, or other oligosaccharide that is 2-DG bound to 2-DG in a 1,4 alpha linkage (2-deoxy-maltose), and that would be cleavable by maltase, and that is 2-DG bound to 2-deoxyfructose to form the 2-deoxy analog of sucrose.

Microtubule inhibitors (as used herein, a “microtubule inhibitor” is any agent that interferes with the assembly or disassembly of microtubules) useful in the practice of the present invention include but are not limited to vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. In accordance with the methods of the present invention 2-DGaI or a 2-DGaI analog is co-administered with a microtubule inhibitor to treat cancer. In one embodiment, the cancer is ovarian cancer, breast cancer, non-small cell lung cancer, Kaposi's sarcoma, and metastatic cancer of breast or ovary origin. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease, lymphosarcoma, reticulum-cell sarcoma, rhabdomyosarcoma, neuroblastoma, and Wilm's tumor. Vincristine is administered in weekly intravenous doses of 2 mg/m² for children and 1.4 mg/m² for adults. In accordance with the methods of the invention, 2-DGaI or a 2-DGaI analog is co-administered with vincristine administered at such doses.

Modifiers useful in the practice of the present invention include but are not limited to Leucovorin (Wellcovorin), which is used with other drugs such as 5-fluorouracil to treat colorectal cancer. In accordance with the methods of the present invention 2-DGaI or a 2-DGaI analog is co-administered with a modifier and another anti-cancer agent to treat cancer. In one embodiment, the cancer is colon cancer. In one embodiment, the modifier is a compound that increases the ability of a cell to uptake glucose, including but not limited to the compound N-hydroxyurea, N-hydroxyurea has been reported to enhance the ability of a cell to uptake 2-deoxyglucose (see the reference Smith et al., 1999, Cancer Letters 141: 85, incorporated herein by reference), and administration of N-hydroxyurea at levels reported to increase 2-DG uptake or to treat leukemia together with administration of 2-DGaI or a 2-DGaI analog as described herein is one embodiment of the therapeutic methods provided by the invention. In another such embodiment, 2-DGaI or a 2-DGal analog is co-administered with nitric oxide or a nitric oxide precursor, such as an organic nitrite or a spernineNONOate, to treat cancer, as the latter compounds stimulate the uptake of glucose and so stimulate the uptake of 2-DGaI or a 2-DGaI analog.

Nitrosoureas useful in the practice of the present invention include but are not limited to procarbazine (Matulane), lomustine, CCNU (CeeBU), carmustine (BCNU, BiCNU, Gliadel Wafer), and estrainustine (Emeyt). In accordance with the methods of the present invention 2-DGaI or a 2-DGaI analog is co-administered with a nitrosourea to treat cancer. In one embodiment, the cancer is prostate cancer or glioblastoma, including recurrent glioblastoma multiforme.

Nucleoside analogs useful in the practice of the present invention include but are not limited to mereaptopurine, 6-MP (Purinethol), fluorouracil, 5-FU (Adrucil), thioguanine, 6TG (Thioguanine), hydroxyurea (Hydrea), cytarabine (Cytosar-U, DepoCyt), floxuridine (FUDR), fludarabine Fludara), pentostatin (Nipent), cladribine (Leustatin, 2-CdA), gemeitabine (Gemzar), and capecitabine (Xeloda). In accordance with the methods of the present invention 2-DGaI or a 2-DGal analog is co-administered with a nucleoside analog to treat cancer. In one embodiment, the cancer is B-cell lymphocytic leukemia (CLL), hairy cell leukemia, adenocarcinoma of the pancreas, metastatic breast cancer, non-small cell lung cancer, and metastatic colorectal carcinoma. As one example, the compound 5-fluoro-2,4(1H,3H)-pyrimidinedione, also commonly known as 5-fluorouracil, is an antimetabolite nucleoside analog effective in the palliative management of carcinoma of the colon, rectum, breast, stomach, and pancreas in patients who are considered incurable by surgical or other means. 5-Fluorouracil is administered in initial therapy in doses of 12 mg/m² given intravenously once daily for 4 successive days with the daily dose not exceeding 800 mg. If no toxicity is observed at any time during the course of the therapy, 6 mg/kg are given intravenously on the 6th, 8th, 10th, and 12th days. No therapy is given on the 5th, 7th, 9th, or 11th days. In poor risk patients or those who are not in an adequate nutritional state, a daily dose of 6 mg/kg is administered for three days, with the daily dose not exceeding 400 mg. If no toxicity is observed at any time during the treatment, 3 mg/kg may be given on the 5th, 7th, and 9th days. No therapy is given on the 4th, 6th, or 8th days. A sequence of injections on either schedule constitutes a course of therapy. In accordance with the methods of the invention, 2-DGaI or a 2-DGaI analog is co-administered with 5-FU administered at such doses or with the prodrug form Xeloda with correspondingly adjusted doses. As another example, the compound 2-amino-1,7-dihydro-6H-purine-6-thione, also commonly known as 6-thioguanine, is a nucleoside analog effective in the therapy of acute non-pymphocytic leukemias. 6-Thioguanine is orally administered in doses of about 2 mg/kg of body weight per day. The total daily dose may be given at one time. If after four weeks of dosage at this level there is no improvement, the dosage may be cautiously increased to 3 mg/kg/day. In accordance with the methods of the invention, 2-DGal or a 2-DGaI analog is co-administered with 6-TG administered at such doses.

Osteoclast inhibitors useful in the practice of the present invention include but are not limited to pamidronate (Aredia). In accordance with the methods of the present invention 2-DGaI or a 2-DGaI analog is co-administered with an osteoclast inhibitor to treat cancer. In one embodiment, the cancer is osteolytic bone metastases of breast cancer, and one or more additional anti-cancer agents are also co-administered with 2-DGaI or a 2-DGaI analog.

Platinum compounds useful in the practice of the present invention include but are not limited to cisplatin (Platinol) and carboplatin (Paraplatin). In accordance with the methods of the present invention 2-DGaI or a 2-DGal analog is co-administered with a platinum compound to treat cancer. In one embodiment the cancer is metastatic testicular cancer, metastatic ovarian cancer, ovarian carcinoma, and transitional cell bladder cancer. As one example, the compound cis-Diamminedichloroplatinum (II), commonly known as cisplatin, is useful in the palliative treatment of metastatic testicular and ovarian tumors, and for the treatment of transitional cell bladder cancer which is not amenable to surgery or radiotherapy. Cisplatin, when used for advanced bladder cancer, is administered in intravenous injections of doses of 50-70 mg/m² once every three to four weeks. In accordance with the methods of the present invention, 2-DGaI or a 2-DGaI analog is co-administered with cisplatin administered at these doses. One or more additional anti-cancer agents can be co-administered with the platinum compound and 2-DGal or a 2-DGal analog. As one example, Platinol, Blenoxane, and Velbam may be co-administered with 2-DGaI or a 2-DGaI analog. As another example, Platinol and Adriamycin may be co-administered with 2-DGal or a 2-DGaI analog.

Retinoids useful in the practice of the present invention include but are not limited to tretinoin, ATRA (Vesanoid), alitretinoin (Panretin), and bexarotene (Targretin). In accordance with the methods of the present invention 2-DGal or a 2-DGaI analog is coadministered with a retinoid to treat cancer. In one embodiment, the cancer is acute promyelocytic leukemia (APL), Kaposi's sarcoma, or T-cell lymphoma.

Topoisomerase 1 inhibitors useful in the practice of the present invention include but are not limited to topotecan (Hycamtin) and irinotecan (Camptostar). In accordance with the methods of the present invention 2-DGal or a 2-DGaI analog is co-administered with a topoisomerase 1 inhibitor to treat cancer. In one embodiment, the cancer is metastatic carcinoma of the ovary, colon, or rectum, or small cell lung cancer. As noted above, however, in one embodiment of the present invention, administration of 2-DGaI or a 2-DGaI analog either precedes or follows, or both, administration of a topoisomerase 1 inhibitor but is not conducted concurrently therewith.

Topoisomerase 2 inhibitors useful in the practice of the present invention include but are not limited to etoposide, VP-16 (Vepesid), teniposide, VM-26 (Vumon), and etoposide phosphate (Etopophos). In accordance with the methods of the present invention 2DGaI or a 2-DGaI analog is co-administered with a topoisomerase 2 inhibitor to treat cancer. In one embodiment, the cancer is refractory testicular tumors, refractory acute lymphoblastic leukemia (ALL), or small cell lung cancer. As noted above, however, in one embodiment of the present invention, administration of 2-DGaI or a 2-DGaI analog either precedes or follows, or both, administration of a topoisomerase 2 inhibitor but is not conducted concurrently therewith.

Tyrosine kinase inhibitors useful in the practice of the present invention include but are not limited to imatinib (Gleevec). In accordance with the methods of the present invention 2-DGaI or a 2-DGaI analog is co-administered with a tyrosine kinase inhibitor to treat cancer. in one embodiment, the cancer is CML or metastatic or unresectable malignant gastrointestinal stromal tumors.

Thus, the present invention provides methods of treating cancer in which 2-DGal or a 2-DGal analog or an acetylated, benzylated, or other modified and prodrug versions thereof and one or more additional anti-cancer agents are administered to a patient. Specific embodiments of such other anti-cancer agents include without limitation 5-methyl-6-[[(3,4,5trimethoxyphenyl)amino-methyl]-2,4-quinazolinediamine or a pharmaceutically acceptable salt thereof, (8S,10S)-10-(3-amino-2,3,6-trideoxy-alpha-L-lyxo-hexopyranosyl)oxy]-8glycoloyl-7,8,9,10-tetrahydro-6,8,11-trihydroxy-1-methoxy-5,2-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]-methylamino]benzoyl]-L-glutamic acid, or a pharmaceutically acceptable salt thereof; or cis-diamminedichloroplatinum (II). The methods of the present invention are generally applicable to all cancers but have particularly significant therapeutic benefit in the treatment of solid tumors, which are characterized by extensive regions of hypoxic tissue. Particular cancers that can be treated with the methods of the present invention are discussed in the following section.

### Treating Particular Cancers

The methods and compositions of the invention may be used to treat any cancer, whether malignant or benign, as well as any precancerous condition, including but not limited to hyperplasias such as benign prostatic hyperplasia. In one important embodiment, the invention provides methods of treating particular types of malignant cancer, including but not limited to non-small cell lung cancer, head and neck cancers, prostate cancer, colon cancer, and breast cancer in humans and other mammals. These methods comprise administering an antineoplastically effective amount of 2-DGal or a 2-DGal analog or a pharmaceutically acceptable salt thereof either alone or in combination with an antineoplastically effective amount of one or more additional anti-cancer compounds. For purposes of illustration and not limitation, the methods of the invention for treating particular types of cancer are described below.

In addition to the cancers listed above, the methods and compositions of the present invention can also be used to treat common cancers such as bladder cancer, colorectal cancer, endometrial cancer, leukemia, lung cancer, lymphoma, melanoma, and ovarian cancer, as well as less common cancers, including but not limited to acute lymphocytic leukemia, adult acute myeloid leukemia, adult non-Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and small-cell lung cancer. Childhood cancers amenable to treatment by the methods and with the compositions of the present invention include but are not limited to brain stein glioma, cerebellar astrocytoma, cerebral astrocytoma, ependyinoma, Ewing's sarcoma and family of tumors, germ cell tumor—extracranial, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, and Wilms's tumor and other childhood kidney tumors.

The methods and compositions of the present invention can also be used to treat cancers that have originated in or metastasized to the bone, brain, breast, digestive and gastrointestinal systems, endocrine system, eye, genitourinary tract, germ cells, gynecological system, head and neck, hematologic system, blood, lung, respiratory system, thorax, musculoskeletal system, and skin.

In one preferred embodiment of the invention, 2-DGaI or a 2-DGal analog is administered to treat non-small-cell lung cancer. Current treatment regiments for non-small-cell lung cancer include without limitation administration of Gemcitabine (Eli Lilly, difluorodeoxycytidine), vinorelbine, paclitaxel, docetaxel, cisplatin, carboplatin, or Irinotecan (camptothecin-11) as single agents; and administration of etoposide and cisplatin, Vindesine (deacetyl vinblastine carboxamide) and cisplatin, paclitaxel and carboplatin, Gemcitabine and carboplatin, docetaxel and cisplatin, vinorelbine and cisplatin, or Irinotecan and cisplatin in combination therapies. See Bunn, 15 Sep. 2002, J. Clin. Onc. 20(18s): 23-33, incorporated herein by reference. In accordance with the methods of the present invention, 2-DGaI or a 2-DGaI analog can be co-administered in such therapeutic regimens to improve patient outcomes.

In another preferred embodiment of the invention, 2-DGaI or a 2-DGaI analog is administered to treat prostate cancer. In one embodiment, 2-DGaI or a 2-DGal analog is administered with prednisone to treat prostate cancer. The present invention also provides pharmaceutical formulations comprising prednisone admixed with 2-DGaI or a 2-DGaI analog in amounts effective for the treatment of prostate cancer and suitable for oral administration. In another embodiment, 2-DGaI or a 2-DGaI analog is administered with prednisone and mitoxanthrone for the treatment of prostate cancer. In another embodiment, 2DGaI or a 2-DGal analog is administered with Taxotere™ (Aventis, docetaxel) for the treatment of prostate cancer.

In another preferred embodiment of the invention, 2-DGaI or a 2-DGaI analog is administered to treat liver cancer, including but not limited to any hepatic tumor, hepatocellular carcinoma, or hepatoma, alone or in combination with other anti-cancer agents. While not to be bound by theory, this aspect of the invention may be particularly effective due to the increased uptake, relative to other cells, of 2-DGaI and certain 2-DGal analogs, including but not limited to 2-F-DGaI and 2-difluoro-DGal, by liver cells (see Keppler et al., 1985, Adv. Enz. Reg. 23: 61-79, supra), and to their retention, once inside the liver cancer cell, due to (i) the presence of galactokinase that converts them to their 1-phosphate derivatives (see Bauer et al., June 1980, Cancer Res. 40: 2026-2032); and (ii) the decreased expression of glucose-6-phosphatase (“G6Pase”) in liver cancer cells (see Ashmore et al., September 1958, Cancer Res. 18(8): 974-979, and Landau et al., November-December 1962, Cancer 15(6): 1188-1196). In one embodiment, the hepatoma to be treated is relatively undifferentiated, and/or the level of G6Pase in the tumor is measured prior to administration of the 2-DGaI or 2-DGaI analog, with the treatment decision based on the amount of G6Pase measured. Tumors that have no or very low levels of G6Pase are deemed to be especially sensitive to treatment with 2-DGaI or a 2-DGaI analog in accordance with the methods of the invention.

In another preferred embodiment of the invention, 2-DGal or a 2-DGaI analog is administered to treat brain cancer, alone or in combination with other anti-cancer agents and/or radiation therapy. While not to be bound by theory, this aspect of the invention may be particularly effective due to the increased uptake, relative to other cells, of 2-DGaI and certain 2-DGaI analogs, including but not limited to 2-F-DGaI and 2-difluoro-DGaI, by brain cells.

In other preferred embodiments of the invention, 2-DGaI or a 2-DGaI analog is administered to treat a cancer selected from the group consisting of cancers arising from leukocytes, fibroblasts, kidney cells, skeletal muscle cells, intestinal mucosa cells, testicular cells, and ovarian cells, alone or in combination with other anti-cancer agents. While not to be bound by theory, this aspect of the invention may be particularly effective due to the increased uptake, relative to other cells, of 2-DGaI and certain 2-DGal analogs, including but not limited to 2-F-DGaI and 2-difluoro-DGaI, by such cells.

In another aspect, the present invention provides a method for determining whether administration of 2-DGaI or a 2-DGaI derivative is effective against a particular tumor or to follow the progress of such therapy, which method comprises subjecting the patient being treated to PET scanning using, for example, either ¹⁸F-2-DG or ¹⁸F-2-DGaI as the imaging agent.

Because the methods of the present invention are applicable to a wide variety of cancers in a wide variety of tissues and organ systems, those of skill in the art will appreciate that the active compound can be formulated in a wide variety of formulations, with the particular formulation selected by the practitioner of skill in the art to best treat the patient. Formulations provided by the present invention are discussed in the following section.

### Formulation and Packaging of 2-DGaI or a 2-DGal Analog

The present invention provides a pharmaceutically acceptable formulation of 2DGaI or a 2-DGaI Useful in the methods of the present invention. In one embodiment, the formulation is crystalline in nature, and the 2-DGaI or 2-DGaI analog is packaged in a sachet that is decanted into a potable liquid for oral administration to the patient. In this embodiment, the liquid can be a syrup or, more conveniently, a commonly consumed liquid, such as water, fruit juice, or cola. In some embodiments, the liquid will be glucose-free. In another embodiment, the 2-DGaI or 2-DGal analog is formulated as a tablet or pill containing 2-DGaI or a 2-DGaI analog in an amount in the range of about 100 mg to about 10g. In some embodiments, each tablet or pill contains about 1g to about 5g of 2-DGaI or a 2-DGal analog.

A decided practical advantage of the compounds of the present invention is that the compounds can be administered in any convenient manner such as by the oral, intravenous, intramuscular, topical, or subcutaneous routes.

Thus, 2-DGaI or a 2-DGal analog can be orally administered, for example, with an inert diluent or with an assimilable edible carrier, including but not limited to any type of food, or it can be enclosed in hard or soft shell gelatin capsules, or compressed into tablets, or incorporated directly with the food of the diet. For oral therapeutic administration, 2-DGaI or a 2-DGaI analog can be incorporated with excipients and used in the form of ingestible tablets, buccal tablets, troches, capsules, elixirs, suspensions, syrups, wafers, and the like. Such compositions and preparations contain enough of the active agent to deliver the therapeutically active doses described above.

The tablets, troches, pills, capsules, and the like may also contain the following: a binder such as gum tragacanth, acacia, corn starch, or gelatin; excipients such as dicalcium phosphate; a disintegrating agent such as corn starch, potato starch, alginic acid, and the like; a lubricant such as magnesium stearate; a sweetening agent such as saccharin; and/or a flavoring agent such as peppermint, oil of wintergreen, or cherry flavoring. When the dosage unit form is a capsule, it can contain, in addition to materials of the above types, a liquid carrier. Various other materials can be present as coatings or to otherwise modify the physical form of the dosage unit. For instance, tablets, pills, or capsules can be coated with shellac. A syrup or elixir can contain the active compound, a sweetening agent, methyl and propylparabens as preservatives, and a flavoring such as cherry or orange flavor. Of course, any material used in preparing any dosage unit form should be pharmaceutically pure and substantially non-toxic in the amounts employed. In addition, the active compound can be incorporated into sustained-release preparations and formulations. The 2-DGal or 2-DGal analog can also be administered parenterally or intraperitoneally. A solution of the active compound can be prepared in water suitably mixed with a surfactant such as hydroxypropylcellulose. Dispersions can also be prepared in glycerol, liquid polyethylene glycols and mixtures thereof, and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and, in final form, must be fluid to the extent that easy syringability exists. It must be stale under the conditions of manufacture and storage and must be preserved against the contaminating action of microorganisms such as bacteria and fungi.

The pharmaceutical forms suitable for topical use include oil and water emulsions and liposomal formulations, as well as lotions, creams, and ointments commonly used for topical administration of drugs. The topical formulation optionally includes one or more additional anti-cancer agents to be co-administered with the 2-DGaI or 2-DGaI analog.

The carrier can be a solvent or dispersion medium containing, for example, water, ethanol, polyol, for example, glycerol, propylene glycol, and liquid polyethylene glycol, and the like, suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique, which yield a powder of the active ingredient plus any additional desired ingredient from previously sterile filtered solution thereof.

As used herein, a “pharmaceutically acceptable carrier” includes any and all solvents, dispersion media, coatings, antibacterial and antifungal agents, isotonic agents, absorption delaying agents, and the like. The use of such media and agents for pharmaceutical active substances is well known in the art. Except insofar as any conventional media or agent is incompatible with the active ingredient, its use in the therapeutic compositions of the invention is contemplated. Supplementary active ingredients can be incorporated into the compositions of the invention.

The present invention also provides slow release forms of 2-DGaI or a 2-DGaI analog. In one embodiment, the slow release form is a pharmaceutical formulation in which the 2-DGaI or a 2-DGaI analog is embedded in or coated by a material from which the 2DGaI or a 2-DGaI analog is released over an extended period of time. The present invention also provides slow release forms of 2-DGaI or a 2-DGal analog in which an acid labile polyethylene glycol (PEG) moiety is attached to the 2-DGaI or a 2-DGaI analog, preferably at the hydroxyl groups at the 4 and 6 positions. Such a slow release form can be readily synthesized by first treating PEG with Des Martin periodinanie and reacting the resulting aldehyde 2-DGaI or a 2-DGaI analog. The resulting compound is a novel compound of the invention, having the structure shown below for 2-DG, with the exception that 2-DGaI or a 2-DGaI analog of choice replaces 2-DG in the structure shown (the structure shown is the 4epimer of the 2-DGaI compound of the invention).

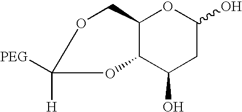

It is essentially advantageous to formulate parental and other compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on the patient and cancer to be treated and can vary from patient to patient and cancer to cancer, but generally, a dosage unit form contains from about 100 mg to about 10g of 2-DGaI or a 2-DGaI analog. Typical unit forms can contain about 0.5 to about 5g of 2-DGaI or a 2-DGaI analog.

The present invention having been described in detail in the preceding sections, the following examples are provided to illustrate certain aspects of, but not to limit the invention.

### EXAMPLE 1

### Growth Inhibitory Activity of 2-Deoxy-D-Qalactose Against Human Heuatocellular Carcinoma Cell Lines

In this example, 2-DGal (or a 2-DGaI analog) can be demonstrated to inhibit cell lines obtained from hepatocellular carcinomas. The cells are cultured at 37 degrees C. in a humidified atmosphere containing 5% CO₂. For passaging, cells grown in 75 cm2 flasks (6070% confluency) are washed with PBS and dissolved from the flasks with trypsin (Gibco BRL) before being plated. Ten thousand cells are plated into each well of a 96-well microtiter plate in 100 ul of medium. The cells are then incubated for 24 h at 37 degrees C. in a humidified atmosphere containing 5% CO₂ prior to exposure to the 2-DG analog or other anti-cancer agent.

Cultures are treated with a range of concentrations of 2-DGaI or a 2-DGaI analog solubilized in cell culture medium, diluted 100× to the desired concentration, continuously for 72 h. Docetaxel (Taxotere™, Aventis Pharmaceuticals, Inc.) solubilized in 100% DMSO and diluted 1000× to the desired concentration, is used as a positive control. The sulforhodamine B (SRB) assay, a dye-based method for determining cell number by virtue of SRB binding to basic amino acids of cellular macromolecules, is employed to assess the growth inhibitory activity of 2-DGal or a 2-DGal analog.

Treatment with 2-DGal or a 2-DGal analog inhibits the growth of hepatocellular 30 carcinoma cell lines. These results demonstrate that liver tumor cells—are sensitive to 2-DGaI or a 2-DGaI analog.

### EXAMPLE 2

### Evaluation of 2-DGaI or a 2-DGaI Analog as a Single Agent and in Combination with Cisplatin

The efficacy of 2-DGaI or a 2-DGaI analog in combination with cisplatin can be compared with cisplatin alone in a tumor growth delay study in a mouse xenograft model. Small pieces of tumor, such as a liver tumor serially passaged in nude mice, are implanted subcutaneously in nude mice, and the tumors allowed to grow to 25 mm². Mice are then randomized to receive no treatment, treatment with 2-DGal or a 2-DGal analog alone, treatment with cisplatin alone, or treatment with both 2-DGal or a 2-DGaI analog and cisplatin. Cisplatin is dosed i.p. at 1 mg/kg on days 1 to 5.2-DGaI or a 2-DGaI analog is dosed orally at 750 mg/kg, twice daily, for the duration of the experiment. Each mouse is sacrificed when its tumor volume reaches 1000 mg.

The results show that treatment with 2-DGal or a 2-DGal analog in combination with cisplatin delays tumor growth significantly more than treatment with cisplatin alone.

### EXAMPLE 3

### Oral Formulations of 2-DGaI or a 2-DGaI Analog

This example illustrates the preparation of representative pharmaceutical formulations for oral administration.

A. 2-DGaI or a 2-DGal analog is dispensed into hard-shell gelatin capsules containing between 100 mg and 1 g of 2-DGal or a 2-DGal analog; optionally, about 0.5% (weight/weight) magnesium stearate can be added. In addition, a mixture of 2-DGaI or a 2-DGal analog and lactose can be used in the capsule.
B. 2-DGaI or a 2-DGaI analog (20.0%-89.9% wt./wt., depending on whether lactose is present, and how much); magnesium stearate (0.9%); starch (8.6%); optionally lactose (0-69.6%) and PVP (polyvinylpyrrolidine; 0.9%) are, with the exception of the magnesium stearate, combined and granulated using water as a granulating liquid. The formulation is then dried, mixed with the magnesium stearate and formed into tablets with a tableting machine.
C. 2-DGal or a 2-DGal analog is dissolved in a mixture of propylene glycol, polyethylene glycol 400, and polysorbate 80; water is added; and the resulting mixture is dispensed into bottles.
D. A mixture of 2-DGal or a 2-DGaI analog (20% to 60% wt./t.), peanut oil (38%-78%), and 2.0% (wt./wt.) Span 60 is prepared, melted, mixed, and filled into soft elastic capsules.

### EXAMPLE 4

### 2-DGaI or a 2-DGaI Analog Formulation for Insufflation

This example illustrates the preparation of a representative pharmaceutical formulation for insufflation. Micronized 2-DGal or a 2-DGaI analog with or without micronized lactose is milled and packaged in an insufflator equipped with a dosing pump.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

### EXAMPLE 5

Liver Toxicity of 2-Fluoro-2-Deoxygalactose

The objective of this study was to evaluate the liver toxicity of 2-Fluoro-2-deoxygalactose when administered via intraperitoneal or intravenous injection to CD 1 mice. The study was initiated on Jan. 11, 2007, and consisted of three groups with three (3) male mice per group. On Day 1, each mouse GROUP was administered either about 0.2 ml of 10% 2 Fluoro 2 Deoxygalactose, intraperitoneally, about 0.2 ml of 10% 2 Fluoro 2 Deoxygalactose, intravenously, or about 0.2 ml of sterile 0.9% sodium chloride for injection, USP, intravenously. Criteria for evaluation included clinical observations and limited serum chemistry parameters (alanine aminotransferase [ALT], aspartate aminotransferase [AST], and total bilirubin). On Day 2, all animals were euthanized, blood was collected, and livers were collected and stored for potential histopathological evaluation.

Clinical observations were performed at least once daily. There were no unscheduled deaths during this study and there were no adverse test article related clinical findings.

B. The issue with the study is that one of the control male animals had very high levels of AST and ALT c/w illness. Based on the historical controls see Table 1, group 1 and animal 2 had an ALT of 421 and an AST of 398. These are the highest of any animals and not consistent with the other two animals in the group receiving sodium chloride. It is believed that this one control animal, a classic CD-1 mouse, was sick and not typical for the strain.
C. The intravenous and intraperitoneal data is very consistent with a treatment effect. The intravenous group, Table 1, group 3 has one animal with elevated enzymes at 24 hours and two that has returned to normal. Given the more limited exposure and clearance of the compound, one would expect this pattern when compared with the intraperitoneal administration where one will have more prolonged exposure, close to IV, but typically a lower Cmax (but above the threshold) but with prolonged exposure. When this group is examined, the data is consistent with galactosamine with a 2-3× rise in ALT and AST over normal animals (based on using the two expected historical controls from Group 1). The data and effects in the intravenous and intraperitoneal group are consistent with expected data and the magnitude of damage at single administration makes sense.
D. The gross pathology finding of a single animal in the intraperitoneal administered group is also c/w a biological effect. It is believed that changes in the control group would not be expected and changes are very unlikely in the intravenous group. In addition, it is believed that there should be a less number of observations in the intravenous group as compared with the intraperitoneal group. It is believed that the fact that there was any damage observed in the intraperitoneal group is a sign consistent with a biological effect.

Study in which 2 fluoro 2 deoxygalactose was administered the CD1 mice intraperitoneally demonstrated increases in concentration of transaminases in accord with the expectation that the compound would damage liver tissue with continued dosing. If there was administration of galactose before providing a deoxy galactose it is believed to be possible to protect normal liver parenchyma while damaging liver tumor (either within the liver itself or for tissue outside of the liver). While studies are not yet completed in terms of specific sites of administration, the procedure outlined derives from the demonstration that galactose markedly inhibits the uptake of 2-Fluoro-2-deoxygalactose by normal liver tissue. An example of the situation would in an individual who has primary liver cancer with or without metastatic disease. This individual would be administered unlabelled galactose to the point where unlabelled galactose reaches the concentration in the hepatic vein that it exceeds the amount that can be absorbed in normal liver. Until that point, the concentration of galactose would be negligible. Thus, we believe to have demonstrated that giving many grams of galactose can be taken up by liver before the liver begins to utilize galactose significantly. Therefore, the subject, for example, receives a treatment of 2 2-Fluoro-2-deoxygalactose at a dose which would normally be taken Lip by liver, but it would not occur if the subject had previously ingested, in the GI tract, high concentrations of galactose. The normal liver would then be protected from 2-Fluoro-2-deoxygalactose. On the other hand since the blood supply to a tumor is arterial the tumor would be exposed to the 2-Fluoro-2-deoxygalactose infused or presented to the tumor tissue. Again, it is believed that the liver would be protected because the galactose given in the 61 tract would compete with the 2 deoxy 2 fluoro galactose that would be attempted to be metabolized by the liver.

TABLE I
Individual Serum Chemistry Values
Lead Optimization Toxicity Study of 2-Fluoro-2-deoxygalactose
Administered Intraperitoneally And Intravenously In CD-1 Mice

| Group | Animal | Day | Alt | Ast | Tbili |
|---|---|---|---|---|---|
| Sex | Number | Number | (U/L) | (U/L) | (mg/dL) |
| 1m | 1 | 2 | 26 | 37 | 0.2 |
| | 2 | 2 | 421 | 398 | 0.3 |
| | 3 | 2 | 32 | 68 | 0.2 |
| 2m | 4 | 2 | 109 | 234 | 0.2 |
| | 5 | 2 | 73 | 114 | 0.3 |
| | 6 | 2 | 72 | 118 | 0.2 |
| 3m | 7 | 2 | 45 | 112 | 0.2 |
| | 8 | 2 | 124 | 116 | 0.2 |
| | 9 | 2 | 30 | 52 | 0.1 |

Nominal Dose: Group 1 - 0.9% Sodium Chloride for Injection, USP, intravenously
Group 2 - 10% 2-Fluoro-2-deoxygalactose, intraperitoneally
Group 3 - 10% 2-Fluoro-2-deoxygalactose, intravenously
KEY FOR SERUM CHEMISTRY VALUES
Alanine aminotransferase (Alt)
Aspartate aminotransferase (Ast)
Total bilirubin (Tbili)

What is claimed as new and desired to be protected by Letters Patent of the United States is:

1. A method of treating or preventing cancer, which method comprises administering to a mammal a therapeutically effective dose of 2-DGal or a 2-DGaI analog.

2. The method of claim 1, wherein said therapeutically effective dose is a dose 2 in the range of about 1 mg/kg (patient weight) to about 1g/kg of 2-DGaI or a 2-DGaI analog.

3. The method of claim 2, wherein said therapeutically effective dose is a dose 2 in the range of about 50 mg/kg to about 250 mg/kg.

4. The method of claim 1, wherein said 2-DGaI or a 2-DGaI analog is administered at least once but not more than four times a day.

5. The method of claim 1, wherein said 2-DGal or a 2-DGaI analog is administered daily for at least three days.

6. The method of claim 5, wherein said 2-DGaI or a 2-DGaI analog is administered daily for at least two weeks.

7. The method of claim 6, wherein said 2-DGal or a 2-DGaI analog is administered daily for at least one month.

8. A pharmaceutically acceptable formulation of a 2-DGal or a 2-DGaI analog and a pharmaceutically acceptable carrier.

9. The formulation of claim 8, wherein said 2-DGal or a 2-DGaI analog is crystalline.

10. The formulation of claim 9, wherein said 2-DGal or a 2-DGal analog is packaged in a sachet.

11. The formulation of claim 8, in which the 2-DGal or a 2-DGal analog is dissolved in a potable liquid.

12. The formulation of claim 11, wherein said liquid is selected from the group consisting of water, fruit juice, and cola.

13. The formulation of claim 8, wherein said 2-DGaI or a 2-DGaI analog is formulated as a tablet or pill containing 2-DGaI or a 2-DGaI analog in the range of about 250 mg to about 2 g.

14. A method of treating or preventing non-small cell lung cancer, which method comprises administering to a mammal a therapeutically effective dose of 2-DGaI or a 2-DGaI analog.

15. A method of treating or preventing cancer, which method comprises administering to a mammal a therapeutically effective dose of 2-DGaI or a 2-DGaI analog in combination with another anti-cancer agent.

16. The method of claim 15, wherein said cancer is a multi-drug resistant cancer or a cancer that is otherwise refractory to treatment.

17. The method of claim 15, wherein said cancer is a cancer selected from the group consisting of non-small-cell lung cancer, head and neck cancel; and breast cancer, and said administering step includes co-administering an antineoplastically effective amount of a compound selected from the group consisting of 5-methyl-6-[[(3,4,5-trimethoxyphenyl)amino]-methyl]-2,4-quinazolinediamine or a pharmaceutically acceptable salt thereof; (8S,1OS)-10-[(3-amino-2,3,6-trideoxy-.alpha.-L-lyxo-hexopyranosyl)oxy]-8-glycoloyl-7,8,9,10-tetrahydro-6,8,11-trihydroxy-1-methoxy-5,12-naphthacenedi one or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]methylamino]benzoyl]-L-glutamic acid, or a pharmaceutically acceptable salt thereof; and cis-diamminedichloroplatinum (II).

18. The method of claim 1 wherein said 2-DGal or a 2-DGaI analog is administered in combination with radiotherapy or surgery.

19. A method of treating liver cancer in a human or other mammal, said method comprising administering to said human or other mammal a therapeutically effective amount 3 of 2-DGaI or a 2-DGaI analog.

20. The method of claim 19, wherein said method further comprises administering one or more additional anti-cancer agents.

21. The method of claim 19, wherein said 2-DGaI or 2-DGaI analog is contained in a disaccharide, trisaccharide, or other oligosaccharide.

22. The method of claim 20, wherein at least one of said one or more additional anti-cancer agents is selected from the group consisting of 2-DG and 3-bromopyruvate.

23. The method of claim 21, wherein said 2-Gal or 2-DGal analog is linked to another monosaccharide in said disaccharide, trisaccharide, or other oligosaccharide by a 1,4 linkage that is cleavable by lactase or maltase.

24. A disaccharide, trisaccharide, or other oligosaccharide comprising 2-Gal or a 2-DGaI analog linked by a 1,4 linkage to another monosaccharide in said disaccharide, trisaccharide, or other oligosaccharide that is cleavable by lactase or maltase.

25. A compound composed of 2-DGaI or a 2-DGaI analog linked to 3-bromopyruvate by an ester linkage.

26. A pharmaceutical composition comprising the disaccharide, trisaccharide, or other oligosaccharide of claim 24 and a pharmaceutically acceptable excipient or carrier.

27. A pharmaceutical composition comprising the compound of claim 25 and a pharmaceutically acceptable excipient or carrier.

28. The compound of claim 24 that is 2-DGal-beta(1,4)-2-DG.
